(19) United States
(12) Patent Application Publication
(10) Pub. No.: US 2022/0240787 A1
(43) Pub. Date: Aug. 4, 2022

(54) SENSOR AGNOSTIC TEMPERATURE DETECTION SYSTEM

(71) Applicant: The United States of America, as represented by the Secretary of the Navy, Crane, IN (US)

(72) Inventors: Aaron Boyd Cole, Bloomington, IN (US); Marcin Stanislaw Malec, Bloomington, IN (US)

(73) Assignee: The United States of America, as represented by the Secretary of the Navy, Arlington, VA (US)

(21) Appl. No.: 17/720,361

(22) Filed: Apr. 14, 2022

Related U.S. Application Data
(63) Continuation-in-part of application No. 17/358,404, filed on Jun. 25, 2021.
(60) Provisional application No. 63/044,091, filed on Jun. 25, 2020.

(51) Int. Cl. A61B 5/01; G01J 5/52

(52) U.S. Cl. CPC A61B 5/01; G01J 5/52

(57) ABSTRACT

Temperature detection systems and methods for detecting temperature of an object are provided. The system utilizes either a predetermined temperature reference by using a well-behaved and calibrated thermal camera with reliable internal temperature reference calibration or one or more calibrated temperature reference devices, such as a blackbody reference, in view of the camera. The system then determines temperature values for each pixel within the image based on mean and median reference temperatures determined from the predetermined temperature reference or the calibrated temperature reference devices and identifies any pixels in the image having a predetermined characteristics, such as those pixels having a temperature greater than the predetermined reference temperature or some other threshold, or, alternatively, pixels in a range of temperatures.

### CROSS-REFERENCE TO RELATED APPLICATIONS

The present application is a Continuation-In-Part of application Ser. No. 17/358,404, filed Jun. 25, 2021, entitled “SENSOR AGNOSTIC TEMPERATURE DETECTION SYSTEM,” the disclosure of which is expressly incorporated by reference herein, which, in turn, claims priority to U.S. Provisional Patent Application Ser. No. 63/044,091, filed Jun. 25, 2020, entitled “SENSOR AGNOSTIC FEBRILE DETECTION SYSTEM,” the disclosure of which is expressly incorporated by reference herein.

### STATEMENT REGARDING FEDERALLY SPONSORED RESEARCH OR DEVELOPMENT

The invention described herein was made in the performance of official duties by employees of the Department of the Navy and may be manufactured, used and licensed by or for the United States Government for any governmental purpose without payment of any royalties thereon. This invention (Navy Case 210071US03) is assigned to the United States Government and is available for licensing for commercial purposes. Licensing and technical inquiries may be directed to the Technology Transfer Office, Naval Surface Warfare Center Crane, email: Cran_CTO@navy.mil.

### FIELD

The field of invention relates generally to temperature sensing and temperature sensors. More particularly, the present disclosure pertains to sensor agnostic temperature detection systems that may be utilized for detection of heat sources, including uses for detecting fevers, tumors, infections, parasites, and the like, as well as for detecting heat of objects such as fire sources for fire detection, fire mitigation, firefighting, and the like.

### BACKGROUND

Rising temperatures can be concerning because they can lead to damage and fires. An aberrant or atypical temperature rise in a system or a component of a system, for example, may often indicate that the system is either malfunctioning or under atypical loads or stresses. Rising temperatures are not only a concern for fires, but also a safety concern for personnel as they might move too close to areas where high temperatures are not usually present and could suffer adverse effects such as burns. Current methods for temperature sensing, measurement, and/or detection may take a relatively long time (i.e., up to a few seconds per reading) and are limited in their accuracy, sensitivity, and precision.

### SUMMARY

The present invention relates to temperature sensing, measurement, and/or detection systems and methods. In an aspect, temperature to be measured is compared to a temperature reference to determine the temperature. The temperature detection systems and methods may be used for detecting human and/or animal fevers, tumors, or infections, parasite infections, environmental heating, and fire source detection, as some examples.

In an aspect, one or more calibrated temperature reference devices are used with an emissive source detector, such as a Mid Wave Infrared (MWIR) thermal camera or a Long Wave Infrared (LWIR) thermal camera that is capable of imaging an object or subject and, in some aspects, one or more calibrated temperature reference devices. Each pixel within the image may be mapped to a specific thermal value based on mean and median reference temperatures, where such mapping identifies the temperature corresponding to each pixel in the image based on the reference temperatures. Additionally, the system may utilize artificial intelligence (AI) or machine learning (ML) (referred to herein as AI/ML) to optimize contrast, filtering errant pixels, and discern other image phenomena or artifacts for obtaining more accuracy of the temperature sensing, measurement, and/or detection. In one aspect, the processed image information may be used to detect an elevated temperature, such as for screening subjects for COVID-19 prior to granting access to a medical facility. In other aspects, the inventive system may be used to identify aberrant temperatures of objects or detect smoke/fire to assist in firefighting.

Additional features, uses, and advantages of the present invention will become apparent to those skilled in the art upon consideration of the following detailed description of the illustrative embodiments for carrying out the invention as presently perceived.

### BRIEF DESCRIPTION OF THE DRAWINGS

The detailed description of the drawings particularly refers to the accompanying figures in which:

FIG. 1 shows a view of one example of calibrated temperature reference devices according to aspects of the disclosure.

FIG. 2 shows a close-up view of an emissive source detector that captures one or more images according to aspects of the disclosure.

FIG. 3 shows a view of a display image with a subject and graphical user interface controls according to aspects of the disclosure.

FIG. 4 shows a diagram of a flow diagram of a temperature detection method according to aspects of the disclosure.

FIG. 5 illustrates another embodiment of a system for detecting temperature according to certain aspects of the disclosure.

FIG. 6 shows yet another embodiment of a system for detecting temperature according to certain aspects of the disclosure.

### DETAILED DESCRIPTION

The embodiments of the invention described herein are not intended to be exhaustive or to limit the invention to precise forms disclosed. Rather, the embodiments selected for description have been chosen to enable one skilled in the art to practice the invention.

Generally, the system is used to detect the surface temperature of an object or subject essentially in real time (e.g., approximately around 0.016 seconds per scan). The temperature information can be used to detect aberrant heat sources (e.g., fires, smoke or overheating objects), as well as to diagnose viral, bacterial, and parasitic infections, including influenza, the common cold, meningitis, urinary tract infections, appendicitis, malaria, and most recently, COVID-19. Additionally, the system can be used for medical detection of tumors. The system can be adapted for use with humans, animals, and plants. Other non-medical uses beyond first detection include oil processing and fuel distillation and chemistry applications where precise temperature measurements are required.

In one embodiment, the system includes one or more black body calibrated temperature reference devices, an emissive source detector that captures one or more images, a user interface (e.g., a display), and a processor running temperature detection software, which will be discussed in greater detail below. The system is Information Assurance/Information Technology (IA/IT) compliant, uses existing hardware, and is system agnostic, which allows use with any emissive source detection sensor. In other aspects, temperature detection may be accomplished using an emissive source detector (e.g., infrared camera) without a calibrated reference device, where the emissive source detector is selected from devices that are well-behaved systems (e.g., linear systems with no or little non-linear behavior) and pixels of the images taken from the device can be accurately correlated to a temperature through a predetermined calibration.

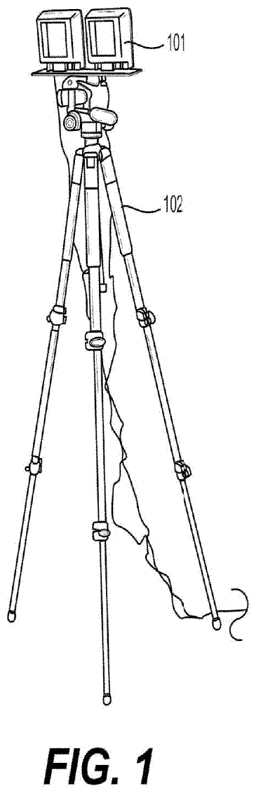

Referring now to FIG. 1, this figure illustrates a view of a portion of an exemplary system including two calibrated temperature reference devices. In this non-limiting example, the reference devices are one or more black body calibrated temperature reference devices 101, such as one or more Body Temperature Reference (BTR) blackbody systems, mounted to a tripod 102. The black body calibrated temperature reference devices 101 provide a stable, uniform, low cost and simple to operate thermal source that serves as an accurate reference for temperature detection. The black body calibrated temperature reference devices 101 provide a viewable thermal reference area for an emissive source detector (and within the field of view of the detector) that captures one or more images (which will be discussed below) to detect the temperature of an object or subject. A reference source is configured as a set point and is stored into non-volatile memory, as one example. After configuration, the black body calibrated temperature reference devices 101 automatically control to (i.e., adjust or revert to) the set point upon each power up.

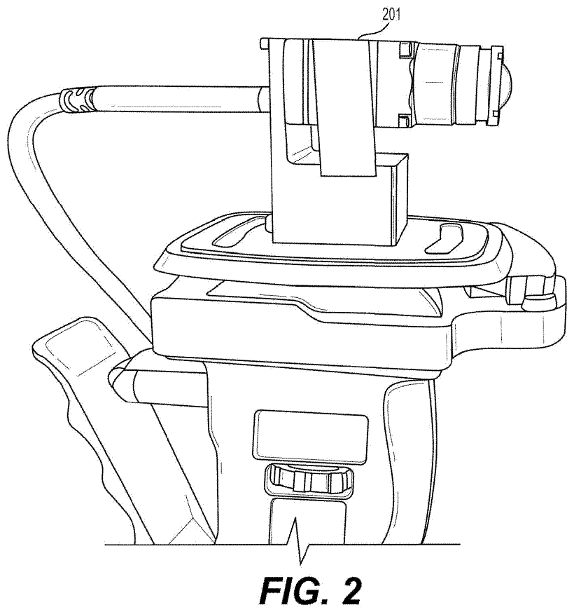

Referring to FIG. 2, this figure illustrates a close-up view of an exemplary emissive source detector (e.g., an infrared thermal camera) that captures one or more images. The emissive source detector preferably comprises either a Mid Wave Infrared (MWIR) thermal camera or a Long Wave Infrared (LWIR) thermal camera 201 (as shown) to perform image capture. These devices generally utilize forward-looking infrared (FLIR) video processing architecture to enable advanced image processing and include multiple industry-standard communication interfaces. The camera 201 provides an infrared or thermal image of a objects or subjects and, in some embodiments, the one or more black body calibrated temperature reference devices 101, which are placed within the field of view of the camera 201, for temperature reference as discussed above. In some examples, the camera device may be a Boson Camera or a Lepton Camera.

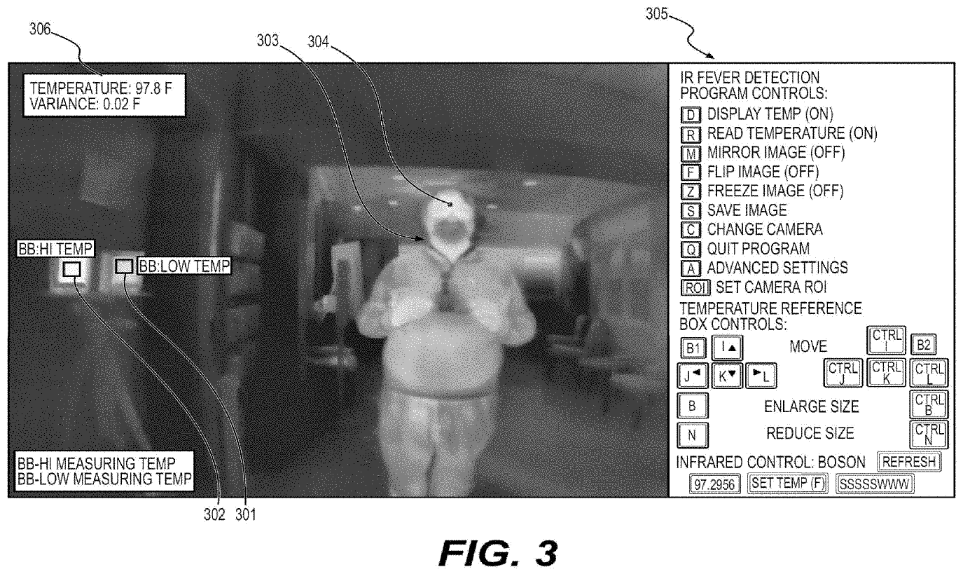

FIG. 3 shows an example of a view of a display image with a subject and exemplary user interface controls, but the disclosure is not limited to this interface display and these user controls. The emissive source detector (e.g., camera 201) captures one or more images and provides an infrared image. Two temperature reference devices 301, 302 are present within the image, with the first temperature reference device 301 set at a low temp reference point and the second temperature reference device 302 set at a high temp reference point. Also within the image is a subject 303 (in this case, a human), but those skilled in the art will appreciate that the system may be applied to detecting temperatures numerous types of other objects as well for a multitude of uses.

The processor, which can be a conventional computer, a specialized processor (e.g., an ASIC), an electronic tablet, a smartphone, or a similar device, executes instructions read from a computer readable memory coupled with the processor in order to receive the one or more images of the subject or object 303 and the two calibrated temperature reference devices 301 and 302 in this particular example. The processor then executes instructions to isolate and analyze the pixel values from the two black body calibrated temperature reference devices 301, 302 to find a mean and a median reference temperature. Next, the processor executes instructions to determine that the mean and median reference temperatures are statistically similar, and uses this information to provide a reference temperature. After determining a reference temperature, the processor executes instructions to map each pixel within the image to a specific thermal value based on the mean and median reference temperatures. Any pixel within the image having a temperature greater than the reference temperature is displayed with a color and/or shape, which allows the elevated temperature to be quickly detected by the system or a trained user.

A set of program controls or graphical user interface (GUI) 305 is provided to operate the system. A region of interest of the subject or object 303 can be easily analyzed by a trained user. As an example, a user can scroll over a desired area with a computer mouse and select an area of the image, such as a human forehead 304 in this particular example, wherein the processor executes instructions to correlate and display the temperature 306 of the desired pixels in the selected area. In the example, the forehead 304 reads a real-time temperature of 98.7° F. with a variance of 0.02° F.

In other aspects, it is noted that the GPU processes may include, but are not limited to, camera control, gain and level control, look up table (LUT) application, heat source monitoring and tracking, metadata control signaling, temperature calculations, image math (e.g., stitch, warp, flip, mirror, resize, de-noising, contours/canny, etc.), and final color LUT.

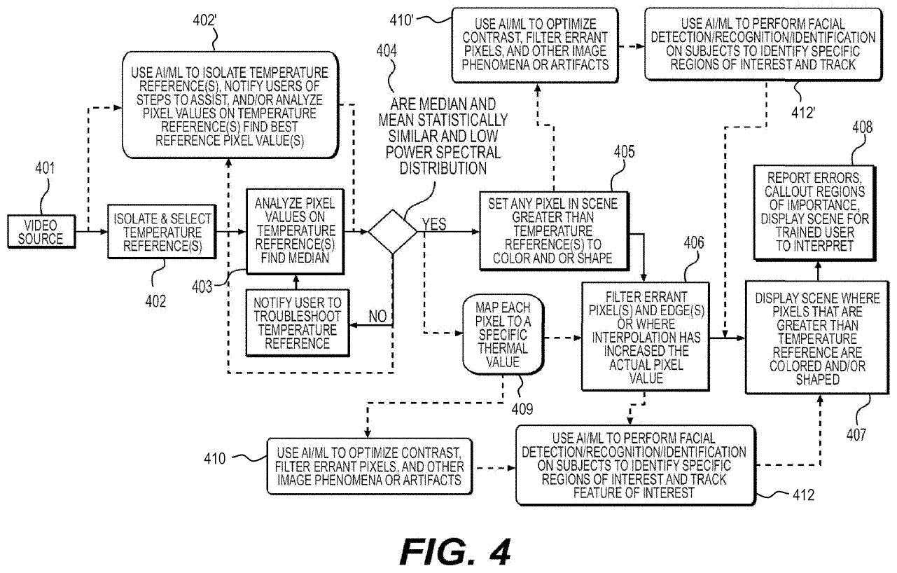

FIG. 4 shows a diagram of a method for determining temperature, which may be implemented by software running on a processor, or through a specialized or dedicated processor (e.g., an ASIC) configured to implement the method of FIG. 4. At 401, images from a video source, such as camera 201, are provided or input. At 402, temperature references are isolated and selected. At 403, pixel values on the temperature reference are analyzed to find the median, as well as to determine the mean. In an alternative or augmented process, AI/ML may be utilized to isolate temperature references, including notification to users of steps to assist or analyze pixel values to find or isolate the best reference pixel values at shown at 402′ and/or find median and mean values of the temperature references.

At a decision block 404, the median and mean temperature references are checked to ensure they are statistically similar. If the means and median are not statistically similar flow may return back to 403 or 402′ to alert a user of needed troubleshooting and to further analyze or isolate temperature references until decision block 404 yields statistical similarity between the median and mean values. In addition, a low power spectral distribution check is performed. At 405, pixels greater than the temperature reference may be set to a specific color and/or shape for ease of identification. Alternatively, each pixel is mapped to a specific thermal value at shown at 409. In either case of blocks 405 or 409, a further alternative includes using AI/ML to optimize the contrast of the image, filter errant pixels, and remove other image phenomena or artifacts that may hinder reading of the thermal data in the image as shown at blocks 410 or 410′.

In still other cases of the flow from blocks 405 or 409, at 406 errant pixels are filtered on edges or where interpolation has increased the actual pixel values. At 407, in some aspects a scene may displayed where pixels that are greater than the reference temperature are shown in the specific color and/or shape. In other aspects, such as for application with heat detection or fire detection of objects, display may not be necessary or the only means of communication of temperature to a user, and further means such as audible alarms or other visual indicators may be utilized instead of or along with display in block 407. In yet further aspects, additional AI/ML may be applied to interpret the image data from block 406 and decide how and what information is displayed, alarmed, or notified.

In further aspects, AI/ML may be used to perform facial or other object detection/recognition/identification of subjects or objects to identify specific regions of interest and/or to track features of interest shown at blocks 412 or 412′ prior to display at 407, which then may include highlighting or emphasizing display of the identified subjects, object, regions of interest, or tracked features. At 408, error reports and callout regions may be produced, and the scene is displayed for interpretation by a trained user.

In another aspect, the temperature detection system and method is used to detect a correlation between temperatures on a subject or object and a temperature reference. The system and method may include one or more calibrated temperature reference devices, such as a first and second BTR, an emissive source detector that captures one or more images, such as a LWIR camera, a display, and a processor in communication with a memory for executing instructions. The processor executes machine readable instructions for performing: receiving one or more images from the emissive source detector, wherein each image includes a subject and the one or more calibrated temperature reference devices; isolating the one or more calibrated temperature reference devices within the image and analyzing pixel values of the one or more calibrated temperature reference devices to find a mean and a median reference temperature; determining that the mean and median reference temperatures are statistically similar, and using the mean and median reference temperatures to provide a reference temperature; mapping each picture within the image to a specific thermal value based on the mean and median reference temperatures; and identifying any pixel in the image with a temperature greater than the reference temperature and displaying the pixels with a temperature greater than the reference temperature with a color and/or shape.

Additionally, a processor may be configured to execute machine readable instructions for filtering errant pixels on edges or where interpolation has increased actual pixel value (See e.g., block 406). Additionally, the processor executes machine-readable instructions for optimizing contrast and filtering errant pixels, image phenomena, and artifacts (See e.g., blocks 410 or 410′). Additionally, the processor executes machine-readable instructions for performing facial or object detection/recognition/identification on the subject to identify and track specific regions of interest (See e.g., blocks 410 or 410′). Additionally, the processor executes machine-readable instructions for displaying errors. Additionally, the processor executes machine-readable instructions for identifying and displaying regions of importance (See e.g., blocks 410 or 410′).

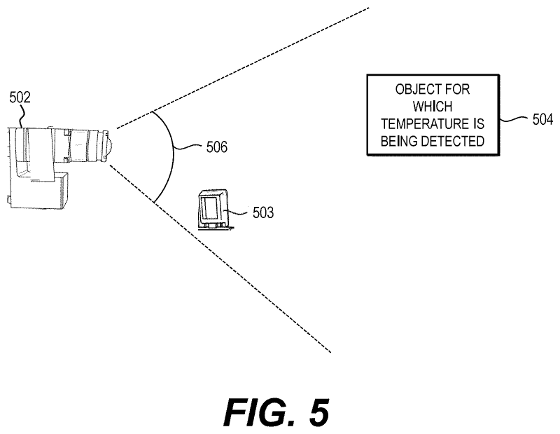

Of further note, the present systems and methods may include the use of simply a single thermal reference device. As an illustration, FIG. 5 shows this scenario including a thermal imaging device 502 and a single reference device 503, which may be similar to device 101 in FIG. 1, but with only a single black body thermal reference. Similar to the case of FIGS. 1-3, an object or subject for which temperature is to be detected 504 is in the view angle 506 of the thermal imaging device 502, along with the single reference device 503. In this scenario, the pixels isolated for the portion of the image including the single reference device 503 (e.g., see block 402 in FIG. 4) are used to establish a single minimum (or maximum) temperature reference, such that when the image is processed, pixels greater than the temperature reference can be delineated (e.g., see block 405 in FIG. 4.)

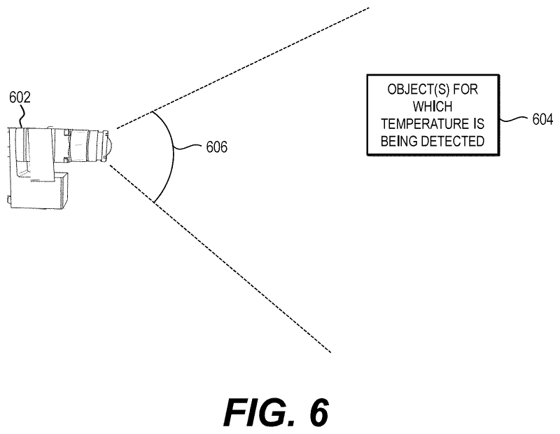

Of still further note, the present systems and methods may include the use of some other temperature reference source without an external reference device such as 101 or 503. As an illustration, FIG. 6 shows this scenario including a thermal imaging device 602 and an object or subject 604 for which temperature is to be detected, which is in the view angle 606 of the thermal imaging device 602. In this scenario, use of a well-behaved system is utilized so that an external reference device such as 101 or 503 is not needed. In particular, a “well-behaved system” is a system with a selected thermal imaging device or camera that does not drift and is stable such that precalibration temperature references that are predetermined before operation are reliable reference temperatures whenever the device or camera is operated.

The systems of FIGS. 4-6 may also then constitute a temperature detection system for detecting temperatures at an object including one or more calibrated reference temperature sources for providing at least one reference temperature, the source comprising one of: (1) a pre-calibrated reference temperature predetermined in an image detection device (e.g., use of the well behaved system including a camera that is stable) or (2) one or more one black body reference devices (e.g., 101 or 503 as discussed above). The system also then includes a thermal image detector or camera that captures one or more images of the object. Further, the system includes a processor in communication with a memory, the processor executing machine readable instruction, where the processor configured to receive one or more images from the image detector, wherein the one or more images include an image of the object, isolate one or more calibrated temperatures using the one or more calibrated reference temperature sources and analyzing pixel values of the one or more calibrated temperature reference sources to find a mean and a median reference temperature, determine that the mean and median reference temperatures are statistically similar, and using the mean and median reference temperatures to provide a reference temperature, and map each pixel within said image to a specific thermal value based on said mean and median reference temperatures.

The gathered data provides a very precise and accurate correlation between the temperatures on the subject and the temperature reference. The system reads temperatures at the frame rate of the imager, meaning temperatures can be calculated in real time (at 0.016 seconds per scan). Compared to currently existing devices that rely on assumptions to assume what the temperature of the subject is, the inventive system and method directly compare the subject's temperature to a known reference temperature, foregoing the need for calculations and dramatically improving accuracy and precision. The data establish a go/no go or pass/fail test for fever rather than wasting time on calculating inaccurate temperatures. The inventive system can simultaneously scan all subjects who fit within the field of view of the sensor in real time, which is a vast improvement over one-at-a-time temperature reading.

The inventive system and method may utilize military sensors, which are by design and legislation much more accurate and robust than medical standoff systems. Military sensors are more adept at detecting very small temperature differences with greater precision and accuracy when compared to consumer off-the-shelf solutions. The inventive system and method are sensor agnostic and can be used with any emissive source detection sensor, such as with MWIR and LWIR. The inventive system can detect any elevated temperature on a human body, which can be used to call attention to other infections or other medical conditions. The inventive system can track, observe, and scan moving subjects from 0 to beyond 600 feet without interference. Additionally, the inventive system can function outdoors as long as the temperature reference and the subject are not in direct sunlight.

In other use cases, the thermal cameras may be adapted to monitor or watch an area for the purposes of fire detection, mitigation, and firefighting, including challenging locations such as submarines. Additionally, it is noted that the dynamic range of the system is customizable such through the use of a display lookup table (LUT), particularly for cases where precision of temperature measurement is less important, such as for detecting aberrant temperatures or fires, or where there is a needs to suppress what is displayed (e.g., suppress display of temperatures below a threshold).

Furthermore, the system may utilize dynamic trackers to reduce some of its precision so that an area may be monitored for aberrant temperatures as well as fires. In operation, the system configured in this manner will operate such that, when a fire breaks out, the system watches and monitors an active fire through most smoke. For example, an LWIR camera can see through most forms of smoke unless that smoke is chemically unique or exceptionally dense. In other aspects, the system may be configured to monitor the temperature of components and areas for temperature increases that could be concerning. For example, rising temperatures (e.g., a particular ΔT) can be of concern because this differential rise in temperature could develop into or cause a fire. Moreover, atypical temperature rises in a system/component, often indicate that the system is either different/malfunctioning or under atypical load/stress.

In yet further aspects, it is noted that the system may be configured to account for privacy and/or safety. For example, in certain areas it is desirable to eliminate or filter out false positives. In such cases, all temperature readings below some predetermined threshold minimum value are ignored to eliminate false positive temperature alerts. Such configuration may also afford privacy. For example, the minimum threshold temperature may be used to monitor crew quarters where privacy is a concern. In those instances for example, all temperatures below approximately around 105° to 120° F. would be forced to display black (i.e., pixels with human forms (and attendant temperatures of humans) picked up by the thermal camera are blacked out) and, thus, people engaged in private activities would not be viewable to a user of the system, but fires and burning contraband would still be obvious in the display.

In yet other aspects, the system may also be configured to discern or differentiate the heat source causing a fire from the surrounding objects that are on fire. This feature would enable firefighters to focus their efforts on where the heat energy is coming from so that when that source is suppressed first, and then the other secondary fires around it can be more easily suppressed (e.g., a battery fire in a room full of combustibles).

In other aspects, the system may include a plurality of cameras, such as an array of cameras, that can be mounted in a facility, a vessel (e.g., a submarine), or other location to monitor a larger area. The cameras, which can be less than one pound in weight, can be attached to surfaces/walls, etc., via various affixing means such as via magnets or Velcro®. Additionally, the GUI could be configured to display shows all cameras simultaneously or to select between individual cameras. Further, a user can configure GUI to show changes, display live video, or display stale video. In other aspects, video frame rate will be greater than 14 FPS may be used. Moreover, temperature may be displayed on objects approaching and surpassing temperature set points (e.g., temperature readout values superimposed on the objects in the display). Further, in some aspect video will be ingested at 14.2/16 bits but displayed at 8 bits with a lookup table applied. Video may be grayscale then color as temperature approaches set point (maximize contrast).

In some other aspects, the presently disclosed systems and method may be applied to animal husbandry (e.g., livestock & herd monitoring for fevers to detect sick animals, detecting skin parasites that raise temperature in a localized area, fevers in cats, dogs, etc.). Other uses may be for wild fire detection, where the system can passively or actively survey for hot spots either caused spontaneously through lightning strikes, solar heat, or from putting out a larger fire. In this case, the system could be used on fire watch towers, aircraft, mobile ground systems, etc.

Yet other uses may include search and rescue to search for missing persons, for example. The advantage of this system over other thermal imagers used for this purpose would be the ability to “fine tune” the desired range and only see temperatures between a certain range and ignore temperatures outside of a human's range.

Yet more uses for the present systems and methods may include food services such as massive food prep, industrial scale (e.g., fast food assembly lines), detecting sick livestock in a processing plant, maintaining temperature in applications where food has to be between temperature maximums and minimums. Other applications may include industrial processes where large kilns, ovens, and furnaces are used and need to be at an exact or “perfect” temperature for melting, forging, etc., ceramics where cones need to be at a perfect temperature range, metals like blacksmithing. Still other applications may include cancer screening (e.g., skin and breast screening where temperatures close to skin surface are easy to detect. If it is unknown if a patient has internal metal such as screws, implants, etc., could be used in conjunction with an MRI to detect as the scanning is happening to prevent damage to surrounding tissue. Also, localized infections might be detected with the system.

Yet further, the system may be applied to electronic equipment monitoring to detect thermal runaway, overheating, etc. Also, in processes such as injection and epoxy molding processes, these processes are very temperature dependent and could be monitored with the present system, as well as paint and other polymers that need to cure.

Additionally, the above-mentioned devices may rely on assumptions or precalibration thresholds to determine the temperature of a subject where the system does not rely on a comparison of the temperature of the subject to a known temperature as discussed above in connection with FIG. 6, for example.

In other examples, the system may utilize physics based models or physics rules for determining the temperature of the pixels in an image. In this case, rules centered around physics/physical phenomena are used in order to reduce the computation load that an artificial intelligence/machine learning (AI/ML) algorithm would otherwise experience. In particular, by ruling out known events and parameters that the computer or processor can ignore, this further enables the AI/ML to focus solely on those fewer technological elements that actually pertain to the data the AI/ML will process. The method of ruling out known noise parameters and errant signals is to set those corresponding data values to something on which the computer or processor will expend minimal to no processing. In this manner, data that is tagged varies based upon code platform and hardware such as: (1) use replacement parameters of setting values to an extreme, like zero or maximum bit value in order to create large magnitudes of contrast within the matrix; (2) setting values to not a number (NaN) so that a particular function ignores the parameter; or isolate areas within a matrix that then becomes a much smaller submatrix for the AUML to work on. The benefit of this approach is computational velocity where the computer or processor only processes what it should and therefore does not waste any resources on extemporaneous and irrelevant data.

Although the invention has been described in detail with reference to certain preferred embodiments, variations and modifications exist within the spirit and scope of the invention as described and defined in the following claims.

1. A temperature detection system for detecting temperatures at an object, the system comprising:
one or more calibrated reference temperature sources for providing at least one reference temperature, the source comprising one of: (1) a pre-calibrated reference temperature predetermined in an image detection device or (2) one or more one black body reference devices;
a thermal image detector that captures images including one or more images of the object; and
a processor in communication with a memory, the processor executing machine readable instruction, the processor configured to:
receive one or more images from the thermal image detector, wherein the one or more images include the one or more images of the object;
isolate one or more calibrated temperatures using the one or more calibrated reference temperature sources and analyzing pixel values of the one or more calibrated temperature reference sources to find a mean and a median reference temperature;
determine that the mean and median reference temperatures are statistically similar, and using the mean and median reference temperatures to provide a reference temperature; and
map each pixel within said image to a specific thermal value based on said mean and median reference temperatures.

2. The system of claim 1, wherein the processor is further configured to identify any pixel in the one or more images from the thermal image detector with a temperature greater than the reference temperature and display those pixels with a temperature greater than the reference temperature with a color and/or shape.

3. The system of claim 1, wherein the one or more one black body reference devices include first and second blackbody devices having respectively different temperature references that enable the system to differentiate between the respective different temperature references.

4. The system of claim 1, further comprising the processor executing machine-readable instructions for performing filtering errant pixels in the one or more thermal images from the thermal image detector on edges or where interpolation has increased actual pixel values.

5. The system of claim 1, wherein the processor is further configured to optimize contrast and filter errant pixels, image phenomena, and artifacts in the one or more images from the thermal image detector.

6. The system of claim 1, wherein the processor is further configured to perform object detection/recognition/identification on said object to identify and track specific regions of interest in the one or more images of the object.

7. The system of claim 1, wherein the processor is further configured to:
compare the specific thermal value for each pixel of the one or more images of the object to the reference temperature; and
provide notification of aberrant thermal conditions of the object when the specific thermal value for each pixel of the one or more images of the object exceed the reference temperature.

8. The system of claim 1, wherein the processor is further configured to display the one or more images and black out pixels of the displayed one or more images that are below a predetermined threshold temperature.

9. A system for detecting temperatures of an object, said system comprising:
at least one processor configured for:
receiving one or more images from a thermal image detector, wherein the one or more images include the one or more images of an object;
isolating one or more calibrated temperatures using the one or more calibrated reference temperature sources and analyzing pixel values of the one or more calibrated temperature reference sources to find a mean and a median reference temperature;
determining that the mean and median reference temperatures are statistically similar, and using the mean and median reference temperatures to provide a reference temperature; and
mapping each pixel within said image to a specific thermal value based on said mean and median reference temperatures.

10. A method for temperature detection system for detecting temperatures at an object, the method comprising:
determining at least one reference temperature comprising one of: (1) a pre-calibrated reference temperature predetermined in an thermal image detector or (2) one or more one black body reference device references;
receive one or more images from the thermal image detector, wherein the one or more images include the one or more images of an object;
isolate one or more calibrated temperatures using the one or more calibrated reference temperature sources and analyzing pixel values of the one or more calibrated temperature reference sources to find a mean and a median reference temperature;
determine that the mean and median reference temperatures are statistically similar, and using the mean and median reference temperatures to provide a reference temperature; and
map each pixel within said image to a specific thermal value based on said mean and median reference temperatures.

11. The method of claim 10, wherein the processor is further comprising:
identifying any pixel in the one or more images from the thermal image detector with a temperature greater than the reference temperature; and
displaying those pixels with a temperature greater than the reference temperature with a color and/or shape.

12. The method of claim 10, wherein the one or more one black body reference devices include first and second blackbody devices having respectively different temperature references that enable the system to differentiate between the respective different temperature references.

13. The method of claim 10, further comprising performing filtering errant pixels in the one or more thermal images from the thermal image detector on edges or where interpolation has increased actual pixel values.

14. The method of claim 10, further comprising optimizing contrast and filtering errant pixels, image phenomena, and artifacts in the one or more images from the thermal image detector.

15. The method of claim 10, further comprising performing object detection/recognition/identification on said object to identify and track specific regions of interest in the one or more images of the object.

16. The method of claim 10, further comprising:
comparing the specific thermal value for each pixel of the one or more images of the object to the reference temperature; and
providing notification of aberrant thermal conditions of the object when the specific thermal value for each pixel of the one or more images of the object exceed the reference temperature.

17. The method of claim 10, wherein the processor is further configured to display the one or more images and black out pixels of the displayed one or more images that are below a predetermined threshold temperature.
